(19) United States
(12) Patent Application Publication
(10) Pub. No.: US 2020/0368554 A1
(43) Pub. Date: Nov. 26, 2020

(54) RADIATION THERAPY SYSTEM, CONTROL METHOD FOR CONTROLLING MEDICAL DEVICE, AND CONTROLLER FOR CONTROLLING MEDICAL DEVICE

(71) Applicant: Regain Biotech Corp., New Taipei (TW)

(72) Inventors: LIANG-HSIANG WU, New Taipei (TW); NGOT-SWAN CHONG, New Taipei (TW); CHIH-CHING CHANG, New Taipei (TW); YI-CHING LIU, New Taipei (TW); MING-HSUN HSU, New Taipei (TW)

(21) Appl. No.: 16/676,058

(22) Filed: Nov. 6, 2019

(30) Foreign Application Priority Data
May 21, 2019 (TW) 108117567

(51) Int. Cl. A61N 5/10

(52) U.S. Cl. CPC A61N 5/1049; A61N 2005/1057

(57) ABSTRACT

A radiation therapy system includes a radiation source, a processor, a flexible pressure sensing matrix, and a pressure sensing device. The radiation source emits a radiation beam to a target region of a patient for treatment. The processor is coupled to the radiation source for adjusting an incident angle and intensity of the radiation beam. The flexible pressure sensing matrix is placed at an object for detecting an amount of movement and an amount of rotation of the object. The pressure sensing device is coupled to the flexible pressure sensing matrix for receiving the amount of movement and the amount of rotation of the object. When the amount of movement or the amount of rotation exceeds a first preset amount, the processor controls the radiation source to suspend treatment.

### FIELD

The subject matter herein generally relates to radiation therapy, and more particularly to a radiation therapy system, a control method for controlling a medical device, and a controller for controlling the medical device.

### BACKGROUND

Generally, before performing radiation therapy, doctors usually take different medical images in advance of a target region for treatment, such as by computerized tomography, MRI images, X-ray films, and the like. The doctors then typically set a treatment plan for the target region to be treated, and the treatment plan is converted into a file that is read by a radiation therapy device. However, a position of the patient may deviate during treatment, which increases risk of damaging normal tissue adjacent to the target region.

### BRIEF DESCRIPTION OF THE DRAWINGS

Implementations of the present disclosure will now be described, by way of embodiments, with reference to the attached figures.

FIG. 1 is a schematic diagram of a headrest coupled to a pressure sensing device.

FIG. 2a is a diagram of an initial position of a patient's head for treatment.

FIG. 2b is a diagram of the patient's head in a rotated state.

FIG. 3a is a diagram of a pressure distribution map according to the initial position in FIG. 2a.

FIG. 3b is a diagram of a pressure distribution map according to the rotated state in FIG. 2b.

FIG. 4 is a schematic block diagram of a pressure sensing device.

FIG. 5 is a schematic block diagram of an embodiment of a radiation therapy system.

FIG. 6 is a schematic block diagram of an embodiment of a controller for controlling a medical device.

FIG. 7 is a diagram showing a patient's head resting on a headrest.

FIG. 8 is a diagram showing a target region of a patient being treated by a radiation source.

FIG. 9 is a flowchart of an embodiment of a control method implemented by a controller for controlling a medical device.

FIG. 10a is a diagram of an initial position of the patient's head and a target region.

FIG. 10b is a diagram of the patient's head rotated from the initial position.

FIG. 11 is a flowchart of an embodiment of a control method implemented by a controller for controlling a medical device.

FIG. 12 is a flowchart of an embodiment of a control method implemented by a controller for controlling a medical device.

### DETAILED DESCRIPTION

It will be appreciated that for simplicity and clarity of illustration, where appropriate, reference numerals have been repeated among the different figures to indicate corresponding or analogous elements. Additionally, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series and the like.

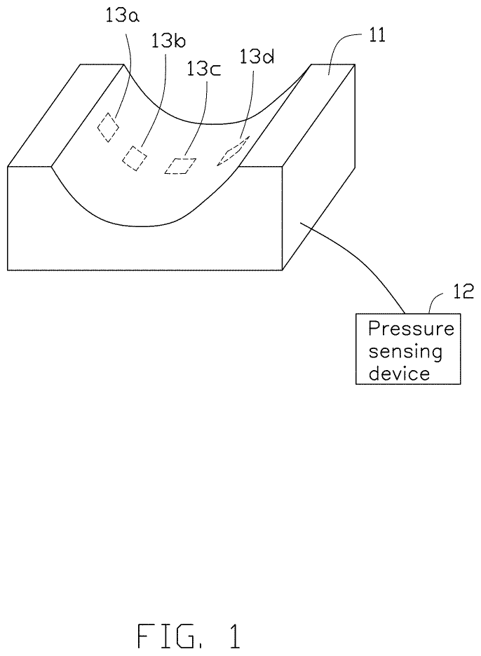

FIG. 1 shows a schematic diagram of a headrest 11 including a flexible pressure sensing matrix (not shown). The headrest 11 is coupled to a pressure sensing device 12. The flexible pressure sensing matrix is disposed on an inner surface of the headrest 11 or in an inner layer of cloth inside the headrest 11. A shape of an inside of the headrest 11 can be designed to fit a patient's head shape, or the headrest 11 can be made of memory foam material. The flexible pressure sensing matrix may include a plurality of pressure sensors, or the flexible pressure sensing matrix may be composed of a plurality of interlaced pressure lines. The pressure sensing device 12 acquires sensed values from the flexible pressure sensing matrix to determine a change in movement or rotation of a patient's head on the headrest 11.

When the patient's head is placed on the headrest 11 in an initial position for treatment, an initial time point is recorded, and sensed values of the flexible pressure sensing matrix are acquired. The sensed values may be pressure values of pressure sensors on the flexible pressure sensing matrix or may be impedance values of the plurality of pressure lines of the flexible pressure sensing matrix. The pressure sensing device 12 continuously or periodically receives the sensed values from the flexible pressure sensing matrix, and whether the patient's head is moving or rotating can be correspondingly determined.

In one embodiment, the pressure sensing device 12 compares the received sensed values to the sensed values at the initial time point according to a comparison table to determine whether the patient's head is moved or rotated beyond a preset amount. If the patient's head is moved or rotated beyond the preset amount, the pressure sensing device 12 transmits a warning signal to a radiation therapy machine through a wireless communication module to suspend therapy.

For example, when the pressure sensing device determines from the received sensed values that the pressure at a region 13a and a region 13b of the flexible pressure sensing matrix increases, and the pressure at a region 13c and a region 13d decreases, the pressure sensing device determines that the head of the patient has been moved or rotated toward the region 13a. The pressure sensing device 12 determines an amount of movement or rotation of the patient's head based on the pressure difference and the comparison table, and determines whether the amount of movement or rotation exceeds the preset amount. In one embodiment, if the amount of movement or rotation is greater than the preset amount, the pressure sensing device 12 transmits a warning signal to the radiation therapy machine through the wireless communication module to suspend therapy. In another embodiment, the pressure sensing device 12 transmits the amount of movement or rotation of the head to the radiation therapy machine, and a control unit of the radiation therapy machine determines whether to suspend therapy.

The pressure sensing device 12 and the headrest 11 are adaptable to a variety of radiation therapy machines through specific communication protocols.

Generally, a human skull is a nearly symmetrical structure. When the patient's head is completely in contact with the flexible pressure sensing matrix and the head moves or rotates, a symmetrical pressure change occurs on the flexible pressure sensing matrix. The regions 13a and 13d are symmetrically disposed on the flexible pressure sensing matrix, and the regions 13b and 13c are symmetrically disposed on the flexible pressure sensing matrix.

When the headrest 11 is made of a hard material, an amount of deformation of the headrest 11 is small, so that a change in horizontal displacement of the patient's head can be ignored. When the patient's head is rotated in the direction of the region 13a, the sensed value at the region 13a (which may be the pressure value or the impedance value) is increased, and the sensed value at the symmetrical region 13d is correspondingly reduced. At the same time, the sensed value sensed at the region 13b increases, and the sensed value at the symmetrical region 13c correspondingly decreases. Further, the amount of increase in the sensed value at the region 13b is higher than the amount of increase in the sensed value at the region 13a. Thus, when the pressure sensing device 12 detects the above-described pressure change, the pressure sensing device 12 determines that the patient's head is rotating.

Next, the pressure sensing device 12 determines an amount of rotation of the patient's head according to the pressure change of the flexible pressure sensing matrix, estimates an amount of displacement of a target region of the head according to the amount of rotation, and determines whether the amount of displacement is higher than a preset amount. If the amount of displacement of the target region is higher than the preset amount, a probability that the treatment may damage normal tissue adjacent to the target region is high, such as 30%-50%, so the radiotherapy machine will suspend treatment so that the position of the patient's head can be re-adjusted.

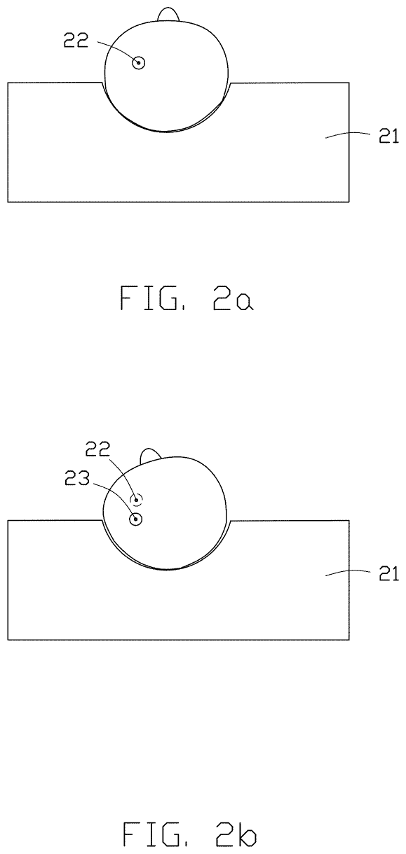

FIG. 2a shows an initial position of the patient's head for treatment. The patient's head is placed on a headrest 21, and a position 22 is a position of a target region of the head for treatment. FIG. 2b shows the patient's head in a rotated state. As shown in FIG. 2b, the target region of the patient's head for treatment is moved from the position 22 to a position 23. At this time, whether to continue or suspend treatment according to a horizontal and vertical displacement of the target region relative to the position 22 is determined.

In the example described above, the pressure sensing device 12 receives the sensed values from the flexible pressure sensing matrix and determines whether the patient's head has moved or rotated according to a change in pressure distribution.

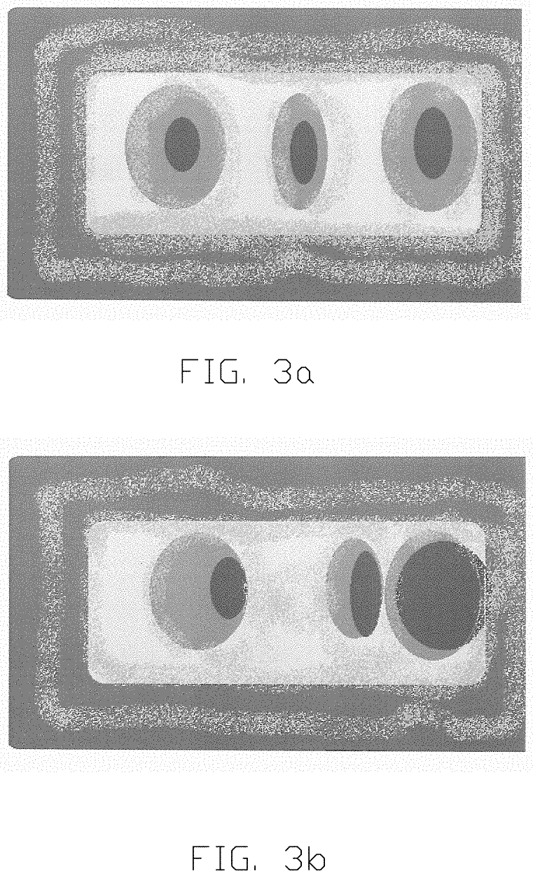

FIGS. 3a-3b show diagrams of a pressure distribution map of the patient's head on the headrest. When the patient's head is in the initial position on the headrest, the pressure distribution in FIG. 3a occurs. When the patient's head has rotated, the pressure distribution in FIG. 3b occurs. The pressure sensing device 12 can determine whether the patient has moved or rotated the head based on changes in the pressure distribution map. When the patient makes a head movement or rotation, the pressure sensing device 12 detects the sensed values from the corresponding regions 13a-13d and calculates whether the amount of displacement of the target region exceeds the preset amount. If the amount of displacement of the target region exceeds the preset amount, the radiation therapy machine is notified to suspend treatment. Thus, the pressure sensing device 12 determines whether to suspend treatment and notifies the radiation therapy machine to suspend treatment.

In another embodiment, the pressure sensing device 12 calculates the amount of displacement of the target region caused by movement or rotation and transmits the amount of displacement to the radiation therapy device, and the radiation therapy device determines whether to suspend treatment.

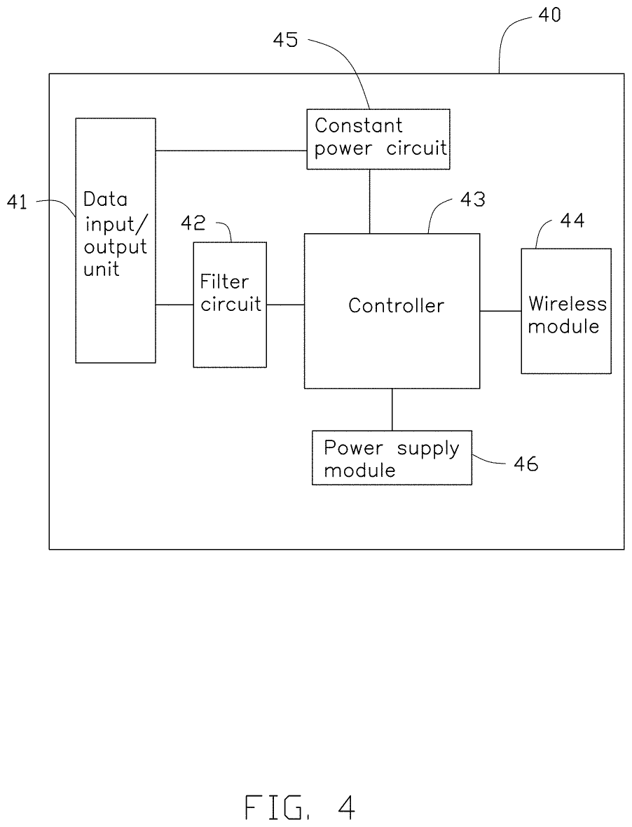

FIG. 4 shows a schematic block diagram of a pressure sensing device 40. The pressure sensing device 40 includes a data input/output unit 41, a filter circuit 42, a controller 43, a wireless module 44, a constant power circuit 45, and a power supply module 46. The data input/output unit 41 is coupled to a flexible pressure sensing matrix, such as the flexible pressure sensing matrix of the headrest in FIG. 1. The constant power circuit 45 transmits a constant voltage or a constant current to the flexible pressure sensing matrix through the data input/output unit 41. The filter circuit 42 receives the sensed value from the flexible pressure sensing matrix through the data input/output unit 41 and filters out noise from the sensed value, and then transmits the filtered sensed value to the controller 43. The controller 43 determines whether the patient's head has moved or rotated beyond the preset amount based on the filtered sensed value. If the patient's head has moved or rotated beyond the preset amount, the controller 43 transmits a warning signal to the radiation therapy device through the wireless module 44. The power supply unit 46 provides the power required by the flexible pressure sensing matrix and the pressure sensing device 40. In one embodiment, the power supply unit 46 is a battery. In another embodiment, the power supply unit 46 is a wireless power receiving device for sensing a change in power or magnetic force in the radiation therapy device to generate the required power. In other words, when the wireless power receiving device is employed, the flexible pressure sensing matrix operates only when the treatment is performed.

In one embodiment, the controller 43 first estimates whether the movement or rotation of the user's head exceeds the preset amount. If the preset amount is exceeded, the controller 43 calculates the amount of displacement of the target region of the head. The displacement includes a horizontal displacement and a vertical displacement. If the amount of displacement exceeds the preset amount, the controller 43 transmits a warning signal to the radiation therapy device to suspend treatment. In one embodiment, the pressure sensing device 40 communicates the preset amount to the radiation therapy device via an encrypted communication protocol.

In one embodiment, the controller 43 first estimates whether the movement or rotation of the user's head exceeds the preset amount. If the preset amount is exceeded, the controller 43 transmits the sensed value to the radiation therapy device through the wireless module 44, and the radiation therapy device calculates the amount of displacement of the target region of the head. The amount of displacement includes the horizontal displacement and the vertical displacement. If the amount of displacement exceeds the preset amount, the radiation therapy device suspends treatment and notifies a healthcare professional to adjust the position of the patient's head.

In one embodiment, the filter circuit 42 is a Kalman filter. The sensed value returned by the flexible pressure sensing matrix has a time stamp. The Kalman filter calculates a current value at the time stamp according to a previous value at a previous time point, and then updates a predictive algorithm according to the actual measured value. The Kalman filter allows the sensed values to be converted into a smoother curve, which reduces noise or common mode noise caused during sensing.

The foregoing embodiments are described with the head as the region of treatment, but can be applied to radiation treatment of a chest cavity, an abdominal cavity, or the like, as long as a size of the headrest is modified. In addition, during thoracic treatment, the flexible pressure sensing matrix can also detect changes in chest breathing to determine when to start radiation therapy and when to suspend radiation therapy.

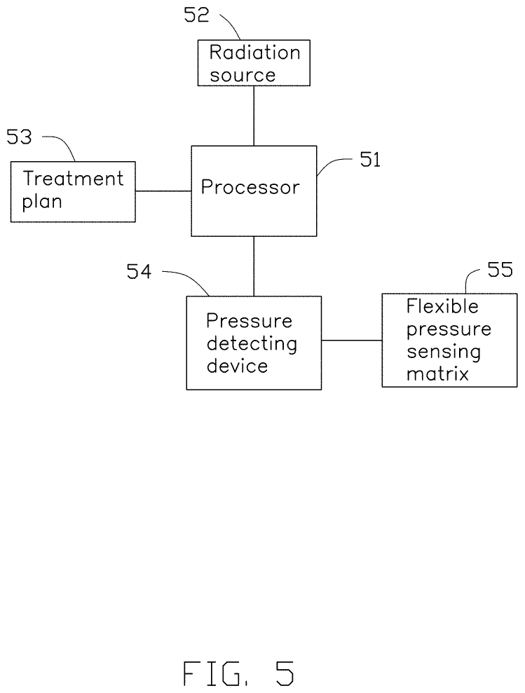

FIG. 5 shows a schematic block diagram of an embodiment of a radiation therapy system. The radiation therapy system includes a processor 51, a radiation source 52, a treatment plan 53, a pressure detecting device 54, and a flexible pressure sensing matrix 55. The treatment plan 53 is stored in a storage device. The processor 51 controls an intensity and direction of radiation applied by the radiation source 52 to the target region for treatment in accordance with the treatment plan 53.

The radiation source 52 emits a radiation beam to the target region. The processor 51 is coupled to the radiation source 52 for adjusting an incident angle and intensity of the radiation beam. The flexible pressure sensing matrix 55 is placed at region of the patient to be treated. The pressure detecting device 54 is coupled to the flexible pressure sensing matrix 55 for detecting an amount of movement or rotation of the region. The processor 51 controls the radiation source 52 to suspend emission of the radiation beam when the amount of movement or rotation exceeds a first preset amount. When the amount of movement or rotation exceeds a second preset amount, the processor 51 controls the radiation source 52 to reduce the intensity of the radiation beam. The second preset amount is within a range of the first preset amount. In one embodiment, the first preset amount of the amount of movement is −5 mm to 5 mm, and the second preset amount of the amount of movement is −2.5 mm to 2.5 mm. In one embodiment, the first preset amount of the amount of rotation is −15 degrees to 15 degrees, and the second preset amount of the amount of rotation is −7.5 degrees to 7.5 degrees.

The pressure sensing device 54 may have a structure as illustrated in FIG. 4. The pressure detecting device 54 may include a constant power circuit (such as the constant power circuit 45), a data input/output unit (such as the data input/output unit 41), a controller (such as the controller 43), a filter circuit (such as the filter circuit 42), a wireless module (such as the wireless module 44), and a wireless power sensing device. The constant power circuit transmits a constant voltage or a constant current to the flexible pressure sensing matrix 55 through the data input/output unit and receives a returned sensed value.

The filter circuit receives the sensed value returned by the flexible pressure sensing matrix through the data input/output unit and filters out noise, and then transmits the filtered sensed value to the controller.

The controller determines, according to the sensed value, whether the amount of movement or the amount of rotation exceeds the first preset amount. The controller transmits the sensed value to the processor 51 through the wireless module, and the processor calculates the amount of movement and/or the amount of rotation and determines whether the amount of movement or the amount of rotation exceeds the first preset amount. The wireless power sensing device senses a change in a magnetic field or an electric field to generate power for the pressure detecting device.

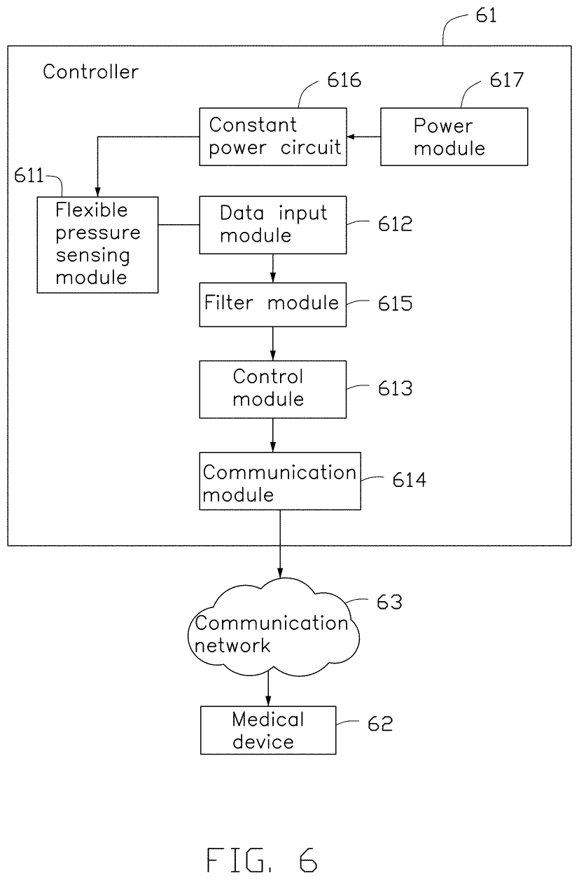

FIG. 6 shows an embodiment of a controller 61 for controlling a medical device 62 through a communication network 63. In one embodiment, the medical device 62 is a radiation therapy device.

The controller 61 includes a flexible pressure sensing module 611, a data input module 612, a control module 613, a communication module 614, a filter module 615, a constant power circuit 616, and a power module 617.

The flexible pressure sensing module 611 includes a plurality of pressure sensors and an electrical circuit connecting the pressure sensors. The pressure sensors are distributed over a plurality of array points of a two-dimensional array. The flexible pressure sensing module 611 electrically couples the constant power circuit 616 and the data input module 612 to receive a constant current or a constant voltage from the constant power circuit 616, and the sensed values generated by the pressure sensors are output to the data input module 612. In another embodiment, the flexible pressure sensing module 611 is composed of a plurality of interlaced pressure lines.

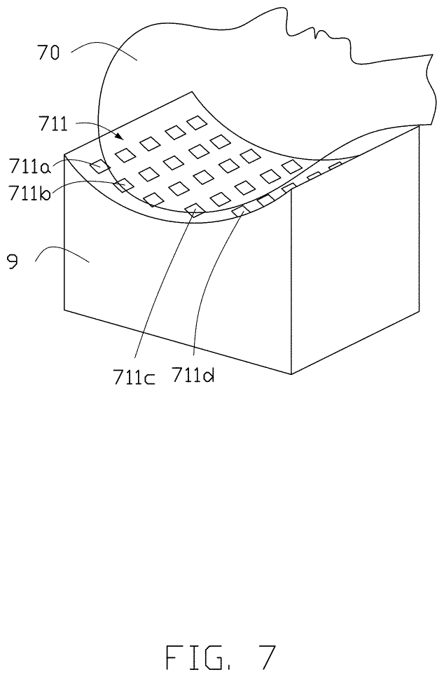

Referring to FIG. 7, in one embodiment, a flexible pressure sensing module 711 (analogous to the flexible pressure sensing module 611) can be disposed on a headrest 7. Pressure sensors 711a to 711d of the flexible pressure sensing module 711 are disposed on an inner surface of the headrest 7 or in an inner layer of cloth inside the headrest 7. A shape of the inside of the headrest 7 can be designed to conform to a shape of the patient's head 70. The patient's head 70 is treated by radiation. The headrest 7 can be made of memory foam material.

The data input module 612 is coupled to the flexible pressure sensing module 611 to receive sensed values from the flexible pressure sensing module 611.

The communication module 614 is coupled to the medical device 62 and transmits the sensed values to the medical device 62 under control of the control module 613. In one embodiment, the communication module 614 supports a particular wireless communication protocol, such as WiFi, BLUETOOTH, or the like. In one embodiment, the communication network 63 includes a wireless communication network.

The filter module 615 is coupled to the data input module 612 and filters out noise of the sensed values received by the data input module 612 and transmits the filtered sensed values to the control module 613. The filter module 615 can be a filtering circuit, such as a Kalman filter. In one embodiment, the sensed values returned by the flexible pressure sensing module 611 have a time stamp. The Kalman filter calculates a current value at the time stamp according to a previous value at a previous time point, and then updates a predictive algorithm or device according to the actual measured value. The Kalman filter allows the sensed values to be converted into a smoother curve, which reduces noise or common mode noise caused during sensing.

The constant power circuit 616 provides one of a constant voltage and a constant current to the flexible pressure sensing module 611.

The power module 617 provides power required by the flexible pressure sensing module 611. In one embodiment, the power supply unit 46 is a battery. In another embodiment, the power supply unit 46 is a wireless power receiving device for sensing a change in power or magnetic force in the radiation therapy device to generate the required power.

The control module 613 is coupled to the filter module 615 and receives data from the data input module 612 through the filter module 615. In one embodiment, the control module 613 receives the sensed values from the flexible pressure sensing module 611, calculates an amount of movement or an amount of rotation of an object on the flexible pressure sensing module 611 (such as the head 70 of FIG. 7) according to the sensed values and a comparison table, determines whether the amount of movement or the amount of rotation is higher than a preset amount, and transmits a warning signal to the medical device 62 when the amount of movement or the amount of rotation is higher than the preset amount to control the medical device 62 to suspend treatment. In one embodiment, the comparison table is stored in the control module 613. In one embodiment, the comparison table describes a relationship between a plurality of pressure differences and a plurality of amounts of movement or rotation. Each pressure difference indicates a plurality of pressure difference values respectively corresponding to the array points. In one embodiment, the controller 61 communicates the preset amount to the medical device 62 via an encrypted communication protocol.

As described above, the flexible pressure sensing module 611 senses a pressure distribution of a contact surface of the headrest 7 that receives the patient's head 70 and generates sensed values of the pressure distribution. The foregoing embodiments are described with the head as the region of treatment, but can be applied to radiation treatment of a chest cavity, an abdominal cavity, or the like, as long as a size of the headrest is modified. In addition, during thoracic treatment, the flexible pressure sensing matrix can also detect changes in chest breathing to determine when to start radiation therapy and when to suspend radiation therapy.

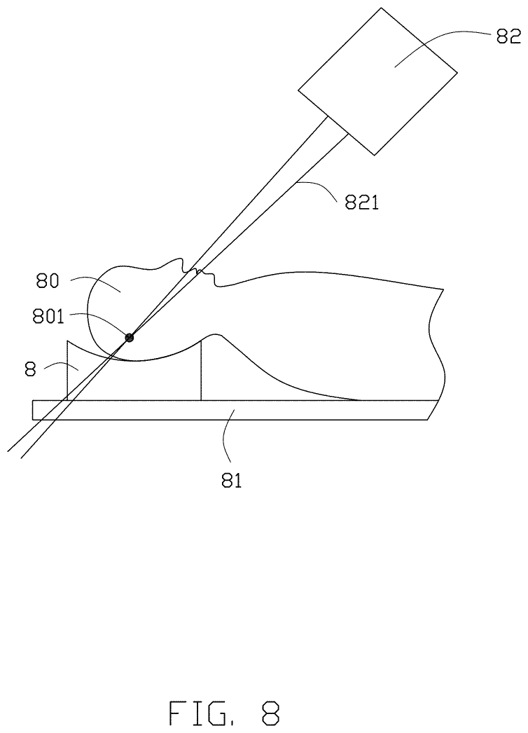

Referring to FIG. 8, in one embodiment when a patient is lying on a platform 81, the patient's head 80 is positioned in an initial position when treatment is initiated. If the head 80 moves such that a position of a target region 801 (a region receiving treatment) of the head 80 deviates from radiation 821 generated by a radiation source 82, normal tissue adjacent to the target region 801 has a risk of being damaged. In this scenario, when the patient's head 80 rests on a headrest 8, the amount of movement or rotation of the head 80 can be measured by the controller 61 (see FIG. 6). Some radiotherapy machines are equipped with cameras to monitor a condition of patients undergoing radiation therapy. Thus, images obtained by the camera can be used in combination with the pressure sensing device during treatment for more accurate treatment.

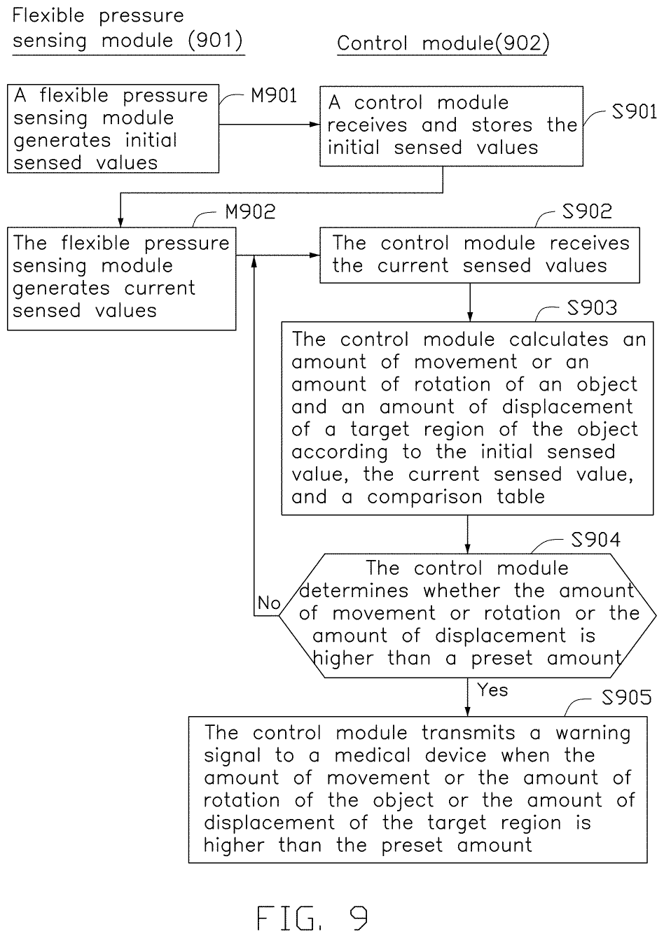

FIG. 9 shows a flowchart of an embodiment of a control method implemented by a controller for controlling a medical device.

At block M901, a flexible pressure sensing module 901 generates initial sensed values. The initial sensed values correspond to an initial position of an object (such as the head 70 of FIG. 7) that rests on the flexible pressure sensing module 901. In one embodiment, the initial sensed values correspond to an initial two-dimensional pressure distribution. For example, referring to FIG. 7, when the head 70 is in an initial position for treatment, each pressure sensor of the flexible pressure sensing module 901 senses pressure of the head 7 on the headrest 7 to produce an initial two-dimensional pressure distribution.

At block S901, a control module 902 receives and stores the initial sensed values from the flexible pressure sensing module 901 via a data input module (such as the data input module 612). For example, when the patient's head is in the initial position, the control module 902 records the sensed pressures from the flexible pressure sensing module 901. In another embodiment, the control module 902 records an impedance of the plurality of pressure lines of the flexible pressure sensing module 901.

At block M902, the flexible pressure sensing module 901 generates current sensed values. The current sensed values relate to a current position of the object (such as the head 80 of FIG. 8). In one embodiment, the current sensed values correspond to a current two-dimensional pressure distribution. In one embodiment, the flexible pressure sensing module 901 continuously or periodically generates sensed values during treatment.

At block S902, the control module 902 receives the current sensed values from the flexible pressure sensing module 901. In one embodiment, during treatment, the control module 902 continuously or periodically accepts the sensed values transmitted by the flexible pressure sensing module 901.

At block S903, the control module 902 calculates an amount of movement or an amount of rotation of the object (such as the head 80 of FIG. 8) and an amount of displacement of the target region (such as the target region 801) according to the initial sensed value, the current sensed value, and the comparison table. In one embodiment, the comparison table is stored in the control module 902.

In one embodiment, whether to continue treatment is determined according to the amount of movement or rotation of the object. In another embodiment, whether to continue treatment is determined according to the amount of displacement of the target region.

When the control module 902 calculates the amount of movement or the amount of rotation of the object and the amount of displacement of the target region at block S903, the control module 902 compares the initial two-dimensional pressure distribution to the current two-dimensional pressure distribution in the comparison table to obtain the amount of movement or the amount of rotation of the object and the amount of displacement of the target region.

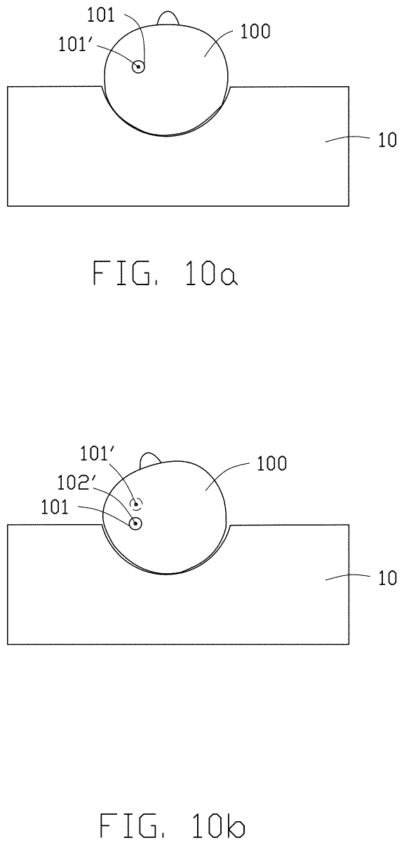

FIG. 10a shows an initial position of the patient's head 100 and a target region 101. FIG. 10b shows the patient's head 100 rotated from the initial position. Because the head 100 is rotated relative to the initial position, the target region 101 is displaced from a position 101′ to a position 102′. The control module 902 first calculates the amount of rotation of the head 100, and then calculates the amount of displacement of the target region 101 based on the amount of rotation. The amount of displacement may refer to a horizontal displacement and a vertical displacement between the position 101′ and the position 102′, that is, the amount of displacement of the target region 101 is described in three dimensions.

At block S904, the control module 902 determines whether the amount of movement or rotation or the amount of displacement is higher than a preset amount. The preset amount is set according to a safety range according to the treatment plan. In one embodiment, the preset amount may correspond to an amount of movement or an amount of rotation of the object (such as the head 30 of FIG. 3). In another embodiment, the preset amount may correspond to an amount of displacement of the target region of the object (such as the target region 301 of FIG. 3).

At block S905, the control module 902 transmits a warning signal to the medical device (the medical device 12) when the amount of movement or the amount of rotation of the object or the amount of displacement of the target region is higher than the preset amount. In one embodiment, the warning signal is used to suspend operation of the medical device to re-adjust the position of the object.

In one embodiment, when the control module 902 calculates the amount of movement or the amount of rotation of the object, the warning signal includes the amount of movement or the amount of rotation, and the medical device determines whether to suspend operation according to the amount of movement or the amount of rotation.

In one embodiment, when the control module 902 calculates the amount of displacement of the target region, the warning signal includes the sensed values from the flexible pressure sensing module 901, such as the initial sensed values and the current sensed values. The medical device calculates the amount of displacement of the target region to be treated (such as the region 801 of FIG. 8) based on the sensed values. When the amount of displacement exceeds the preset amount, the medical device suspends treatment and notifies medical staff to adjust the position of the patient.

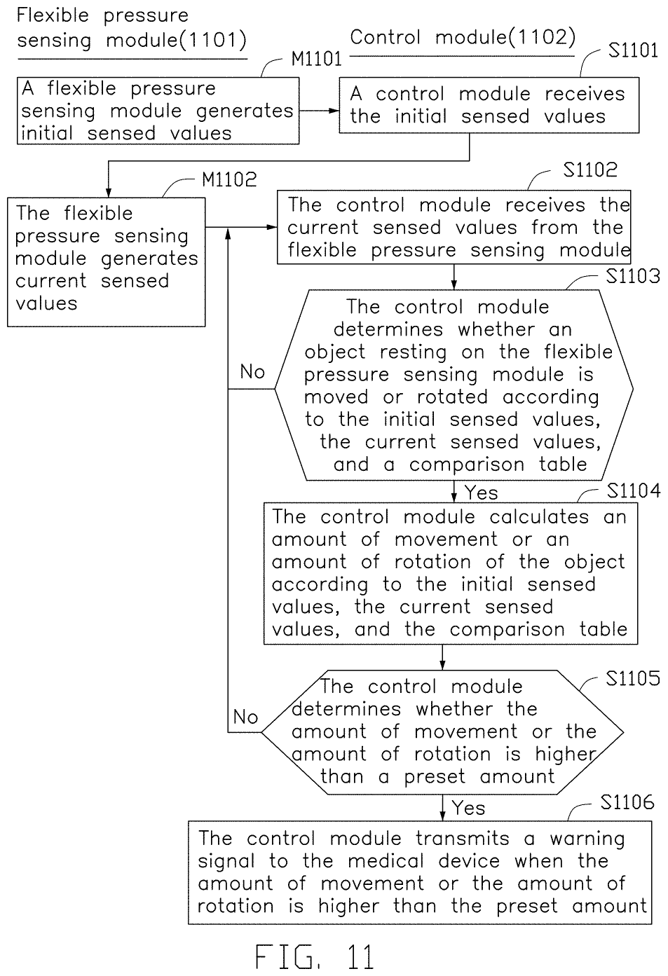

FIG. 11 shows a flowchart of an embodiment of a control method implemented by a controller for controlling a medical device.

At block M1101, a flexible pressure sensing module 1101 generates initial sensed values.

At block S1101, a control module 1102 receives the initial sensed values from the flexible pressure sensing module 1101.

At block M1102, the flexible pressure sensing module 1101 generates current sensed values.

At block S1102, the control module 1102 receives the current sensed values from the flexible pressure sensing module 1101.

At block S1103, the control module 1102 determines whether an object resting on the flexible pressure sensing module 1101 is moved or rotated according to the initial sensed values, the current sensed values, and a comparison table. In general, the human skull is a nearly symmetrical structure, so that when a patient's head is completely in contact with the flexible pressure sensing module and is moved or rotated, pressure changes of the pressure sensors have a specific pattern.

Referring to FIG. 7, for example, when the headrest 2 is made of a hard material, an amount of deformation is small, so that a change in horizontal displacement of the patient's head can be ignored. When the patient's head is rotated in the direction of the region 13a, the sensed value at the region 13a (which may be the pressure value or the impedance value) is increased, and the sensed value at the symmetrical region 13d is correspondingly reduced. At the same time, the sensed value sensed at the region 13b increases, and the sensed value at the symmetrical region 13c correspondingly decreases. Further, the amount of increase in the sensed value at the region 13b is higher than the amount of increase in the sensed value at the region 13a. Thus, when the pressure sensing device 12 detects the above-described pressure change, the pressure sensing device 12 determines that the patient's head is rotating. A pressure change pattern corresponding to the rotation can be stored in the comparison table.

In one embodiment, the control module 1102 makes the determination based on the comparison table and the sensed values received from the flexible pressure sensing module 1101. In another embodiment, the control module 1102 makes the determination based on the pressure distribution map generated by the control module 1102 based on the sensed values.

At block S1104, the control module 1102 calculates an amount of movement or an amount of rotation of the object according to the initial sensed values, the current sensed values, and the comparison table.

At block S1105, the control module 1102 determines whether the amount of movement or the amount of rotation is higher than a preset amount.

At block S1106, when the amount of movement or the amount of rotation is higher than the preset amount, the control module 1102 transmits a warning signal to control the medical device to suspend treatment.

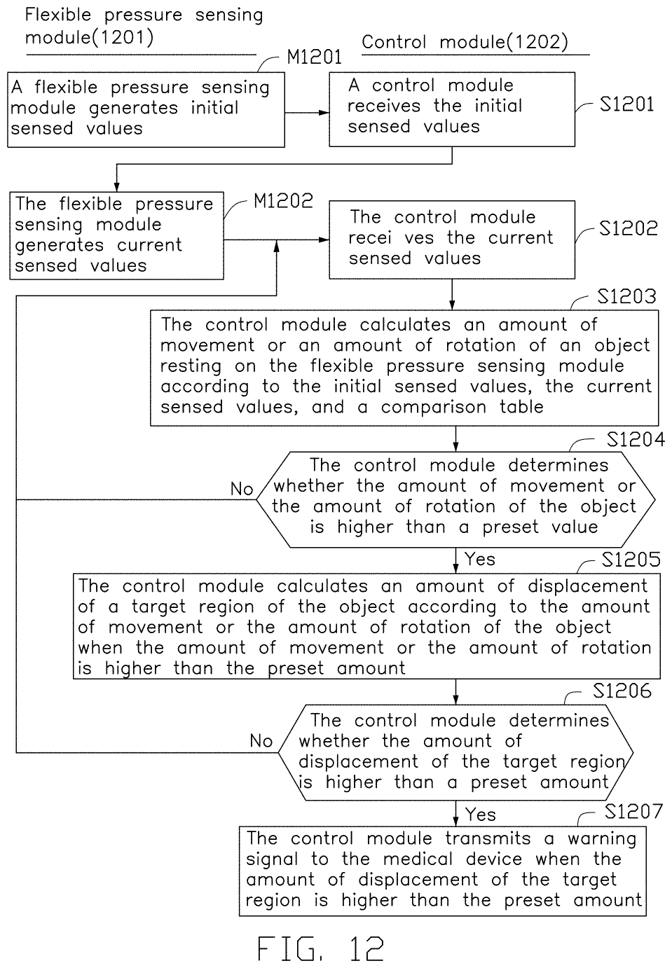

FIG. 12 shows a flowchart of an embodiment of a control method implemented by a controller for controlling a medical device.

At block M1201, a flexible pressure sensing module 1201 generates initial sensed values.

At block S1201, a control module 1202 receives the initial sensed values from the flexible pressure sensing module 1201.

At block M1202, the flexible pressure sensing module 1201 generates current sensed values.

At block S1202, the control module 1202 receives the current sensed values from the flexible pressure sensing module 1201.

At block S1203, the control module 1202 calculates an amount of movement or an amount of rotation of an object resting on the flexible pressure sensing module 1201 according to the initial sensed values, the current sensed values, and a comparison table.

At block S1204, the control module 1202 determines whether the amount of movement or the amount of rotation of the object is higher than a preset value.

At block S1205, when the control module 1202 determines that the amount of movement or the amount of rotation is higher than the preset amount, the control module 1202 calculates an amount of displacement of a target region of the object according to the amount of movement or the amount of rotation of the object.

At block S1206, the control module 1202 determines whether the amount of displacement of the target region is higher than a preset amount.

At block S1207, the control module 1202 transmits a warning signal to the medical device to control the medical device to suspend treatment when the amount of displacement of the target region is higher than the preset amount.

In summary, the positions of the object (70/80/100) and the target region (801/101) are detected by the flexible pressure sensing module (611/901/1101/1201), and a movement or rotation of the object and/or displacement of the target region are calculated according to sensed values from the flexible pressure sensing module. A warning signal is transmitted to the medical device 62 when the amount of movement or the amount of rotation of the object and/or the amount of displacement of the target region is higher than a preset amount to control the medical device 62 to suspend treatment.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

What is claimed is:

1. A radiation therapy system comprising:
a radiation source emitting radiation beams to a target region;
a processor coupled to the radiation source for adjusting an incident angle and intensity of the radiation beam;
a flexible pressure sensing matrix adapted for placing on an object for detecting an amount of movement and an amount of rotation of the object; and
a pressure sensing device coupled to the flexible pressure sensing matrix for receiving the amount of movement and the amount of rotation of the object, wherein:
when the amount of movement or the amount of rotation exceeds a first preset amount, the processor controls the radiation source to stop emitting radiation beams.

2. The radiation therapy system of claim 1, wherein:
when the processor determines that the amount of movement or the amount of rotation exceeds a second preset amount, the processor controls the radiation source to reduce an intensity of the radiation beam; and
the second amount is within a range of the first amount.

3. The radiation therapy system of claim 1, wherein:
the pressure sensing device comprises a constant power circuit, a data input/output unit, and a controller;
the constant power circuit transmits a constant voltage or a constant current to the flexible pressure sensing matrix through the data input/output unit and receives returned sensed values from the flexible pressure sensing matrix.

4. The radiation therapy system of claim 3, wherein:
the pressure sensing device further comprises a filter circuit;
the filter circuit receives the sensed values from the flexible pressure sensing matrix through the data input/output unit and filters out noise from the sensed values, and then transmits the filtered sensed values to the controller.

5. The radiation therapy system of claim 3, wherein:
the controller determines whether the amount of movement or the amount of rotation exceeds the first preset amount according to the sensed values.

6. The radiation therapy system of claim 3, wherein:
the pressure sensing device further comprises a wireless module;
the controller transmits the sensed values to the processor through the wireless module; and
the processor calculates the amount of movement or the amount of rotation and determines whether the amount of movement or the amount of rotation exceeds the first preset amount.

7. A control method for controlling a medical device, the method comprising:
receiving, by a control module, initial sensed values from a flexible pressure sensing matrix;
receiving, by the control module, current sensed values from the flexible pressure sensing matrix; and
determining whether an object on the flexible pressure sensing matrix has moved or rotated, wherein
if the object has moved, calculating, by the control module according to the initial sensed values and the current sensed values and according to a comparison table, an amount of movement or an amount of rotation of the object or an amount of displacement of a target region of the object; and
transmitting a warning signal to the medical device to suspend treatment when the amount of movement or the amount of rotation of the object or the amount of displacement of the target region exceeds a preset amount.

8. The control method of claim 7, wherein:
the initial sensed values correspond to an initial two-dimensional pressure distribution;
the current sensed values correspond to a current two-dimensional pressure distribution; and
the comparison table represents a relationship between a plurality of two-dimensional pressure differences and a corresponding plurality of amounts of movement or rotation.

9. The control method of claim 7, wherein:
the warning signal comprises the amount of movement or the amount of rotation of the object and/or the amount of displacement of the target region.

10. A controller for controlling a medical device, the controller coupled to the medical device and a flexible pressure sensing module, the controller comprising:
a data input module receiving sensed value from the flexible pressure sensing module; and
a control module coupled to the data input module; wherein:
the control module receives initial sensed values and current sensed values from the flexible pressure sensing module through the data input module;
the control module calculates an amount of movement or an amount of rotation of an object on the flexible pressure sensing module or an amount of displacement of a target region of the object according to the initial sensed values and the current sensed values and according to a comparison table;
the control module determines whether the amount of movement or the amount of rotation of the object or the amount of displacement of the target region exceeds a preset amount; and
the control module transmits a warning signal to the medical device when the amount of movement or the amount of rotation of the object or the amount of displacement of the target region exceeds the preset amount.

11. The controller of claim 10, wherein:
the initial sensed values correspond to an initial two-dimensional pressure distribution;
the current sensed values correspond to a current two-dimensional pressure distribution; and
the comparison table represents a relationship between a plurality of two-dimensional pressure differences and a corresponding plurality of amounts of movement or rotation.

12. The controller of claim 10, further comprising a communication module coupled to the control module, the communication module communicatively coupled to the medical device, wherein:
the communication module is controlled by the control module to transmit the warning signal to the medical device.

13. The controller of claim 10, further comprising a filter module coupled to the data input module and the control module, wherein:
the filter module filters out noise from the sensed values received by the data input module.

14. The controller of claim 10, further comprising a constant power circuit electrically coupled to the flexible pressure sensing module, wherein:
the constant power circuit transmits a constant voltage or a constant current to the flexible pressure sensing module.

15. The controller of claim 10, wherein:
the flexible pressure sensing module comprises a plurality of pressure sensors distributed over a plurality of array points of a two-dimensional array.

16. The controller of claim 10, wherein:
the warning signal comprises the amount of movement or the amount of rotation of the object and/or the amount of displacement of the target region.
